(19) United States
(12) Patent Application Publication
(10) Pub. No.: US 2016/0120689 A1
(43) Pub. Date: May 5, 2016

(54) Nasal Dilator

(71) Applicant: Anthonius Fredericus Maria BENDE, Amsterdam (NL)

(72) Inventor: Anthonius Fredericus Maria Bende, Amsterdam (NL)

(21) Appl. No.: 14/890,706

(22) PCT Filed: Apr. 23, 2014
(86) PCT No.: PCT/EP2014/058263

§ 371 Date: Nov. 12, 2015

(30) Foreign Application Priority Data
May 13, 2013 (NL) 1040207
Mar. 6, 2014 (NL) 1040720

(51) Int. Cl. A61F 5/56; A61M 29/00

(52) U.S. Cl. CPC A61F 5/56; A61M 29/00

(57) ABSTRACT

The invention relates to a nasal dilator produced from soft silicone, which nasal dilator comprises two tube-shaped elements each adapted to be positioned within a nostril of a human nose, which tube-shaped elements have each a longitudinal axis, are open on each longitudinal end such that air can pass through the tube-shaped elements, and which tube-shaped elements have each a protrusion on the outer surface adjacent to one end of the tube-shaped element.

### TECHNICAL FIELD

The invention relates to a nasal dilator. This invention is based on the medical field particularly in the field of ENT (ear nose throat).

### BACKGROUND FOR DEVELOPMENT

A number of (approximately 35%) of the people above the 40 years, suffers daily or weekly from snoring, in combination with sleep apnea, more in particular Obstructive Sleep apnea (OSA), which is due to bad breathing caused due to congestion or obstruction of the nostrils by breathing through the nose. The phenomena “mouth breathing” (a wrong way of breathing) is the medical world more and more linked to all kind of severe illnesses like bad oxygen transport, high blood pressure etc.

At this moment there are a number of nasal dilators on the market and on hand, but they all are dealing with a certain percentage of un-comfortability. This often results in a loss of the nasal dilator out the nose during the night, what makes that the user will start again with snoring, with mouth breathing, and almost always followed by dangerous sleep apnea, while he or she is on the assumption that he or she is sleeping healthy, as they are using a nasal dilator. Most nasal dilators are intended to avoid the nostrils from collapsing, the main cause of difficulty breathing through the nose. (Blocked upper airways)

The un-comfortability of nasal dilators in general, is caused by the size and model of the nose/nostril versus the size, shape and/or the model of the nasal dilators. This typically always a problem, as there is not one nose or nostril in the same model. The momentarily existing nasal dilators, are shaped holding the nasal dilator in place as for example “clamped to the nasal septum”, what is painful. An “over sized model” gives a continued pressure on the inside of the nostril. “Pushing outwards models” to hold the nasal dilator in place give an irritating tension on “one” spot of the skin inside the nostril.

Even metal or hard plastic cones placed on one area of the skin are uncomfortable as the materials are too stiff and hard for daily use. This kind of nasal dilators will all result in a tension in the nose/nostrils, making that the highly sensitive mucous membrane, in the inside of the nostril, will be irritated after some hours. Because of that, the irritation will make that the user unconsciously while sleeping, will push or rub “out” the dilator during their sleep, making the intended effect of the dilator disappear. This makes that the user, unknown, will start with snoring again, followed by dangerous or unhealthy sleep apnea “Obstructive sleep apnea (OSA)”

The object of the invention is to reduce or even remove the above mentioned disadvantages.

This object is achieved according to the invention with a nasal dilator produced from silicone, which nasal dilator comprises two tube-shaped elements each adapted to be positioned within a nostril of a human nose, which tube-shaped elements have each a longitudinal axis, are open on each longitudinal end such that air can pass through the tube-shaped elements, and which tube-shaped elements have each a protrusion on the outer surface adjacent to one end of the tube-shaped element.

The comfort-ability of the nasal dilator according to the invention is based on the use of the free space in the “Nose-Tips” of the human nose. From nature, most human noses are provided with a free nose tip in the very front of the nose. Research provided the information that not only European or Western noses that are a bit more “flat”, but all human noses, regardless of race or nature have this free nose tip. The nose tip is foremost empty (some hair will grow in there) and depending on the size of the nose, the nose tip has an empty free space of between the 16 and 6 mm. in depth what can be used for this nasal dilator according to the invention.

In combination with the depth upwards in the nostril, the nasal dilator will be accommodated in the nostril without any pressure needed, because the protrusion will extend into the free space in the nose tip of the human nose and lock the nasal dilator according to the invention in place.

The nasal dilator will thus “rest” or “hang” on the edge of the bottom or lower part of the nose-tip by the protrusion, while the tube-shaped elements are resting to the back of the nostril. These tube-shaped elements are slightly on the side and tilted to ensure the right airstream to inhale. In this situation the nasal dilator will stay in place without any pressure occurring.

This nasal dilator according to the invention avoids mouth breathing in the most comfortable way, while people sleeping, as they are not aware of their own performing of breathing actions in that time of sleeping, what can have an impact on their wellbeing in general but especially on their overall health.

The tube-shaped elements can be used to avoid desiccate of the nostrils, to one and a half, centimeter in the nostril what will/can develop by inhaling dry air, like in air conditioned area's, like planes, sleeping rooms, using CPAP, etc. Due to the contact of the barrier of silicone against the sensitive mucous membrane, the feeling of dried out nostrils will take longer, or will disappear.

Especially, when oxygen is administered to a patient, the tube-shaped elements protect the inner walls of the nose and prevent that these walls desiccate and possibly rupture.

The nasal dilator according to the invention must be made from silicone, what makes the nasal dilator unbreakable and more durable and more cost-effective in time, compared to any other hard plastic or metal nasal dilator, nasal dilator strip etc., as it can go for a lifetime in general.

Furthermore, the silicone material provides for sufficient flexibility, while be able to keep the nostrils open, and reduces the chances on irritation of the nostrils.

In an embodiment of the nasal dilator according to the invention the protrusion on the outer surface of each tube-shaped element extends in a direction having at least a radial component.

In a preferred embodiment of the nasal dilator according to the invention the protrusion declines in longitudinal direction towards the center of the tube-shaped element.

The space in a nostril reduces from the external opening towards the forehead of a person, more or less accordingly with the shape of the nose. Similarly, the space in the nose tip also reduces in the direction of the forehead. Now by having the protrusion declining in longitudinal direction towards the center of the tube-shaped element, it is ensured that the widest part of the protrusion can be accommodated in the nose tip and provide a locking action, while the reducing part will follow the reduction in space of the nostril towards the forehead.

In yet another embodiment of the nasal dilator according to the invention the outer surface of the tube-shaped element is provided with at least two, parallel circumferential grooves on the end opposite of the end with the protrusion for adjusting the length of the tube-shaped elements.

The circumferential grooves provide means to adjust the length of the tube-shaped elements to adjust to the depth of the nostrils. By having rounded edges on the grooves, it is ensured that comfort remains with an shortened tube-shaped element inserted into the nostril.

The nasal dilator according to the invention can also be produced in four or six or more sizes, to provide the user the best fitting size. This production can be done in a for this produced well known manner using a “pressure/vulcanization mold”, or even using an injection mold.

Yet another preferred embodiment of the nasal dilator according to the invention comprising a connection strip, wherein each end of the connection strip is attached to one of the two tube-shaped elements.

The connection strip ensures that the two tube-shaped elements are kept together at all times and that one of the tube-shaped elements of the nasal dilator cannot get lost.

In a further preferred embodiment of the nasal dilator according to the invention the outer surface of each tube-shaped element is provided with a soft sticky like gel, silicone layer.

The nasal dilator according to the invention can be produced from a soft type of silicone as the protrusion holds the nose dilator in the nose in place. A hard or strong metal or plastic part is not needed anymore to provide pressure.

As due to the fact silicone can/must be used, now today, we also can produce “gel-like sticky” silicone for the nasal dilator, that provides an even more steadiness in placement with an even more security of avoiding “slip or rubbing” out. This sticky silicone can be used, but is not always necessary to use. Production of the nasal dilator with a soft sticky like gel, silicone layer is advised for the use by sporting, because the airflow is many times stronger than during sleeping.

This way the type of “gel-like sticky” silicone is not always necessary is this invention, but can be used when required what will increase the comfort approximately 10%/20% more.

On the other hand the cost of production using the gel-like sticky silicone will increase as it must be built in layers. Even tough, this embodiment will bring down the overall costs compared to a breakable alternative, or nasal strips, as the nasal dilator according to the invention can go for many years or even a lifetime what reduces the costs for a device such as the nasal dilator in general.

The surface of the silicone nasal dilator can be cleaned with water and soap for reuse. The extra flexible and or sticky silicone can be produced up to approximately 3 to 7 or higher (when needed) shore skin, in combination to 30 to 60% shore base, depending on the size and thickness of the tube. The nose dilator is build in layers, to ensure the right stiffness, to work as a nasal dilator, following the contours from each individually nose shape.

In still a further embodiment of the nasal dilator according to the invention the tube-shaped elements are substantially cylindrical.

According to the difference in western and Asian shape noses, the substantially cylindrical tube-shaped elements could have a planar wall portion, which extends over the length of the tube-shaped element. The planar wall part follows the nasal septum to guarantee the highest comfort.

Because the tube-shaped elements of the nasal dilator are slightly tilted due to the location inside the nostrils, the tube-shaped elements are exactly in the radius needed for an optimal free breathing. Direct flow in the entrance in the back of the nose, using a nasal dilator according to the invention, on the tube-shaped elements that are preferably tapering from the end provided with the protrusion towards the opposite end, making it possible to use a soft tube-shaped element that barely will touch the inside of the nostril, making that the tube-shaped element hardly can be felt, while the nasal dilator will stay in place, and by eventually unconscious rubbing the nose, always will stay in place for the whole night, while sleeping.

The tube-shaped elements can be also constructed of two or more layers or using inserts, to achieve a higher point of comfort, due to using extra soft silicone at the surface layer, while the insert, can be treat with many different impregnations, like protectors against virus, bacteria, and molds, by inhaling air.

More general, the nasal dilator according to the invention comprises the combination of a nasal dilator with two adjustable tube-shaped elements, connected with a flexible connection, performed with a protrusion and which is made of soft silicone with a sticky gel like surfacing, to achieve the highest comfort for the user, with the ability to stay the whole night in place, what avoids back sleeping, snoring, and Sleep apnea (OSA).

These and other features of the invention will be elucidated in conjunction with the accompanying drawings.

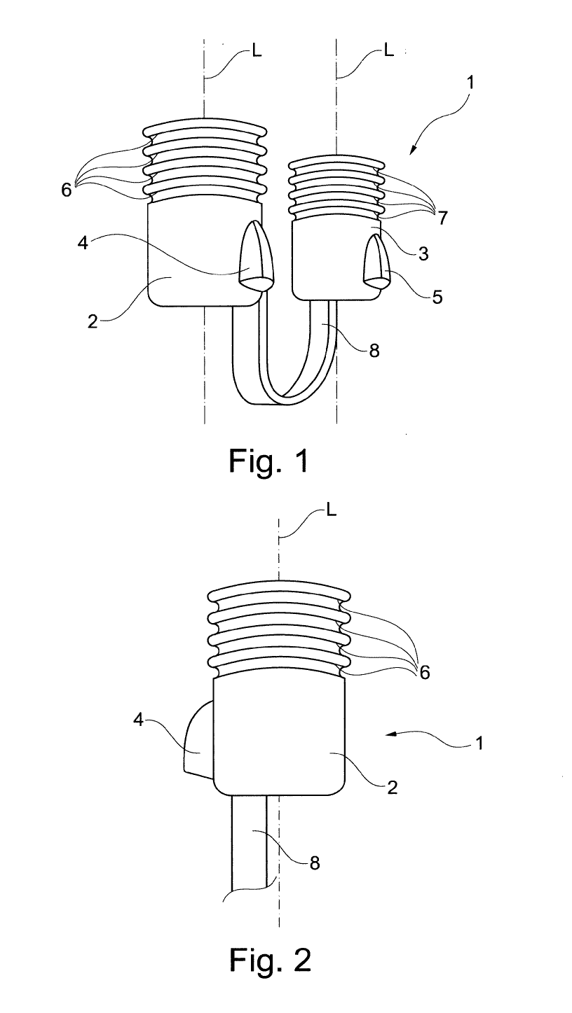

FIG. 1 shows a perspective view of a first embodiment of the nasal dilator according to the invention.

FIG. 2 shows a second perspective view of the embodiment of FIG. 1.

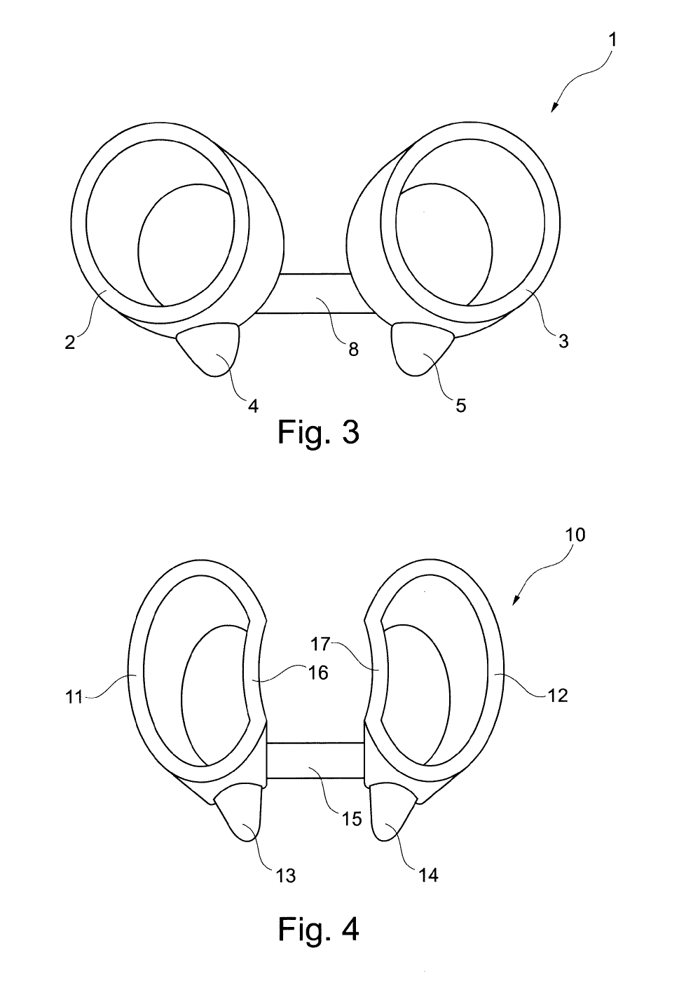

FIG. 3 shows a perspective top view of the embodiment of FIG. 1.

FIG. 4 shows a perspective top view of a second embodiment of the nasal dilator according to the invention.

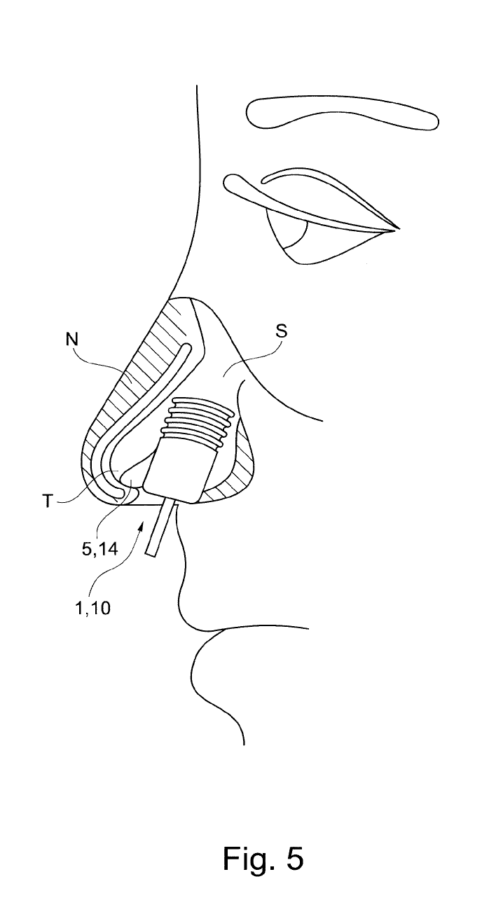

FIG. 5 shows a schematic view of the positioning of a nasal dilator according to the invention in a nose of a person.

In FIG. 1 we can see a first embodiment of the complete nasal dilator 1 according to the invention. The nasal dilator 1 has two tube-shaped elements 2, 3. Each tube-shaped element 2, 3 is provided is provided with a protrusion 4, 5 which declines in longitudinal direction L towards the center of the tube-shaped element 2, 3. (see also FIGS. 2 and 3)

At the end opposite of the end with the protrusions 4, 5 a number of parallel circumferential grooves 6, 7 are provided. These grooves 6, 7 (or the rounded ribs in between) provide a clear position where the tube-shaped elements can be cut in order to shorten the length of the tube-shaped elements 2, 3.

A flexible connection strip 8 is provided, which strip 8 is each with an end attached to a tube-shaped element 2, 3 to keep the elements 2, 3 together.

In FIG. 4. we can see a second embodiment of the nasal dilator 10 according to the invention. The nasal dilator 10 has also two tube-shaped elements 11, 12 provided with a protrusion 13, 14. The two tube-shaped elements 11, 12 are connected together via a flexible strip 15.

In contrast to the substantially cylindrical tube-shaped elements 2, 3 of the embodiment according to FIGS. 1-3, the tube-shaped elements 11, 12 of the nasal dilator 10 has a planar wall portion 16, 17, which extends over the length of the tube-shaped element. This nasal dilator 10 with planar wall parts is in particular designed for a western model nose, or for a divided or over sensitive nostril. The substantial cylindrical element with planar wall part 16, 17 will follow the sensitive parts of the nasal septum, what is in western nose models, often flat.

In FIG. 5 we can see the nasal dilator 1, 10 of the invention positioned in the nostril space S of the nose N of a person. The protrusion 5, 14 extends into the depth of the nose tip T. Here we can see the nasal dilator 1, 10 is sitting “free’ in the nose N using the depth of Nose tip T, avoiding from slip out. The top of the nasal dilator 1, 10 is slightly tilted backwards while the protrusion 5, 14 is placed in the nose tip T.

Some advantages of the nasal dilator according to the invention are:

1. A nasal dilator produced from soft Silicone with connection strip, to be placed in both nostrils in the human nose, to retain the model of the nostril and avoiding obstruction due to under pressure of the nostril by inhaling and exhaling air.

2. A nasal dilator as mentioned in claim 1. Provided with on the front of the tube(s) a nose lock, that can be placed “free” in the “nose tip”, for extreme comfort while “Locking” the nasal dilator in the nostril/vestibule, avoiding the nasal dilator from “falling or rubbing out” unconscious out of the nose while sleeping.

3. A nasal dilator as mentioned in claims 1 and 2, provided with a soft sticky like gel, silicone layer, for extra comfort and strong stability used by sport of sleep, providing extra comfort.

1. A nasal dilator produced from silicone, which nasal dilator comprises two tube-shaped elements each adapted to be positioned within a nostril of a human nose, which tube-shaped elements have each a longitudinal axis, are open on each longitudinal end such that air can pass through the tube-shaped elements, and which tube-shaped elements have each a protrusion on the outer surface adjacent to one end of the tube-shaped element.

2. The nasal dilator according to claim 1, wherein the protrusion on the outer surface of each tube-shaped element extends in a direction having at least a radial component.

3. The nasal dilator according to claim 2, wherein the protrusion declines in longitudinal direction towards the center of the tube-shaped element.

4. The nasal dilator according to claim 1, wherein the outer surface of the tube-shaped element is provided with at least two, parallel circumferential grooves on the end opposite of the end with the protrusion for adjusting the length of the tube-shaped elements.

5. The nasal dilator according to claim 1, further comprising a connection strip, wherein each end of the connection strip is attached to one of the two tube-shaped elements.

6. The nasal dilator according to claim 1, wherein the outer surface of each tube-shaped element is provided with a soft sticky like gel, silicone layer.

7. The nasal dilator according to claim 1, wherein the tube-shaped elements are substantially cylindrical.

8. The nasal dilator according to claim 7, wherein the substantially cylindrical tube-shaped elements have a planar wall portion, which extends over the length of the tube-shaped element.

9. The nasal dilator according to claim 7, wherein the tube-shaped elements are tapering from the end provided with the protrusion towards the opposite end.
